(19) United States
(12) Patent Application Publication
(10) Pub. No.: US 2024/0249452 A1
(43) Pub. Date: Jul. 25, 2024

(54) METHODS AND SYSTEMS FOR ATTENUATION MAP GENERATION

(71) Applicant: SHANGHAI UNITED IMAGING HEALTHCARE CO., LTD., Shanghai (CN)

(72) Inventors: Songsong TANG, Shanghai (CN); Jianxun WANG, Shanghai (CN); Yilin LIU, Shanghai (CN); Yun DONG, Shanghai (CN)

(73) Assignee: SHANGHAI UNITED IMAGING HEALTHCARE CO., LTD., Shanghai (CN)

(21) Appl. No.: 18/437,172

(22) Filed: Feb. 8, 2024

Related U.S. Application Data
(63) Continuation of application No. PCT/CN2022/122717, filed on Sep. 29, 2022.

(30) Foreign Application Priority Data
Sep. 30, 2021 (CN) 202111162422.5

(51) Int. Cl. G06T 11/00; G06T 5/70; G06T 7/00

(52) U.S. Cl. CPC G06T 11/005; G06T 5/70; G06T 7/0012; G06T 2207/10104; G06T 2207/20081; G06T 2211/452

(57) ABSTRACT

A method for attenuation map generation may include obtaining first PET data relating to first background events occur during a first PET scan, which is performed by a PET scanner with no subject within a detection tunnel of the PET scanner; obtaining second PET data relating to second background events occur during a second PET scan, which is performed by the PET scanner with a subject within the detection tunnel; generating, based on the first PET data and the second PET data, a target attenuation map of the subject, wherein the first background events and the second background events may occur when a first particle and a second particle are detected by crystal units of the PET scanner within a background coincident window, and the first particle and the second particle may be different types of particles produced by a radioactive decay of a crystal material of the PET scanner.

### CROSS-REFERENCE TO RELATED APPLICATIONS

This application is a Continuation of International Application No. PCT/CN2022/122717, filed on Sep. 29, 2022, which claims priority to Chinese Patent Application No. 202111162422.5, filed on Sep. 30, 2021, the contents of which are incorporated herein by reference in their entirety.

### TECHNICAL FIELD

The present disclosure relates to the field of medical imaging, in particular, to methods and systems for attenuation map generation.

### BACKGROUND

Positron emission tomography (PET) has been widely used in clinical diagnosis and/or treatment. Before a PET scan, a radioactive tracer may be injected into a subject. A positron generated by the decay of the radioactive tracer may travel within the subject and interact with an electron in the subject. The positron and the electron may annihilate, thus producing a pair of gamma photons with opposite propagation directions. A detector of a PET scanner may detect the pair of gamma photons. The position of the annihilation may be estimated based on a detection time difference of the pair of gamma photons, and a distribution map of the radioactive tracer in the subject may be reconstructed based on the estimated position. In order to improve the quality of the reconstructed PET image, physical correction, including attenuation correction and scattering correction, may need to be performed. Both the attenuation correction and the scattering correction may be performed based on an attenuation map of the subject. The quality and the generation efficiency of the attenuation map may affect the quality and the reconstruction efficiency of the PET image.

Therefore, it is desirable to provide effective methods and systems for attenuation map generation.

### SUMMARY

One aspect of the present disclosure may provide a method for attenuation map generation. The method may include: obtaining first positron emission tomography (PET) data relating to first background events occur during a first PET scan, the first PET scan being performed by a PET scanner with no subject within a detection tunnel of the PET scanner; obtaining second PET data relating to second background events occur during a second PET scan, the second PET scan being performed by the PET scanner with a subject within the detection tunnel; and generating, based on the first PET data and the second PET data, a target attenuation map of the subject, wherein the first background events and the second background events may occur when a first particle and a second particle are detected by crystal units of the PET scanner within a background coincident window, and the first particle and the second particle may be different types of particles produced by a radioactive decay of a crystal material of the PET scanner.

In some embodiments, generating, based on the first PET data and the second PET data, a target attenuation map of the subject may include generating, based on the first PET data and the second PET data, a preliminary attenuation map, the preliminary attenuation map being an attenuation map of the subject with respect to gamma rays with a first energy level; and obtaining the target attenuation map based on the preliminary attenuation map and a conversion coefficient, the target attenuation map being an attenuation map of the subject with respect to gamma rays with a second energy level.

In some embodiments, generating, based on the first PET data and the second PET data, a preliminary attenuation map may include generating a first sinogram based on the first PET data; generating a second sinogram based on the second PET data; generating a difference sinogram between the first sinogram and the second sinogram; and generating the preliminary attenuation map by performing an image reconstruction on the difference sinogram.

In some embodiments, the method may further include: obtaining a first attenuation coefficient of a reference material with respect to gamma rays with the first energy level; obtaining a second attenuation coefficient of the reference material with respect to gamma rays with the second energy level; and determining, based on the first attenuation coefficient and the second attenuation coefficient, the conversion coefficient.

In some embodiments, generating, based on the first PET data and the second PET data, a target attenuation map of the subject may include generating first target PET data by performing a first noise reduction operation on the first PET data; generating second target PET data by performing a second noise reduction operation on the second PET data; and generating, based on the first target PET data and the second target PET data, the target attenuation map of the subject.

In some embodiments, at least one of the first noise reduction operation and the second noise reduction operation may be performed based on a noise reduction model.

In some embodiments, the noise reduction model may be generated using a plurality of training samples, and each of the plurality of training samples may include first sample PET data corresponding to a first acquisition duration and second sample PET data corresponding to a second acquisition duration, wherein the first acquisition duration and the second acquisition duration may be different.

In some embodiments, the first particle may include a beta electron, the second particle may include a gamma photon.

In some embodiments, each coincident event of the first background events and the second background events may be determined by obtaining the background coincident window, the background coincident window including a first energy window corresponding to the first particle, a second energy window corresponding to the second particle, and a time window; determining whether the first particle and the second particle meet a predetermined condition based on a first energy of the first particle, a first detection time of the first particle, a second energy of the second particle, a second detection time of the second particle, and the background coincident window; and in response to determining that the first particle and the second particle meet the predetermined condition, determining that the background event occurs.

In some embodiments, the method may further include obtaining third PET data relating to coincident events occur during a third PET scan, the third PET scan being performed by the PET scanner on the subject who has been injected with a radioactive tracer; and generating, based on the target attenuation map and the third PET data, a PET image of the subject.

Another aspect of the present disclosure may provide a system for attenuation map generation. The system may include at least one storage device storing a set of instructions; and at least one processor configured to communicate with the at least one storage device, wherein when executing the set of instructions, the at least one processor is directed to perform operations including: obtaining first positron emission tomography (PET) data relating to first background events occur during a first PET scan, the first PET scan being performed by a PET scanner with no subject within a detection tunnel of the PET scanner; obtaining second PET data relating to second background events occur during a second PET scan, the second PET scan being performed by the PET scanner with a subject within the detection tunnel; and generating, based on the first PET data and the second PET data, a target attenuation map of the subject, wherein the first background events and the second background events may occur when a first particle and a second particle are detected by crystal units of the PET scanner within a background coincident window, and the first particle and the second particle may be different types of particles produced by a radioactive decay of a crystal material of the PET scanner.

Another aspect of the present disclosure may provide a non-transitory computer readable medium. The non-transitory computer readable medium may include at least one set of instructions, wherein when executed by at least one processor of a computing device, the at least one set of instructions causes the computing device to perform a method. The method may include obtaining first positron emission tomography (PET) data relating to first background events occur during a first PET scan, the first PET scan being performed by a PET scanner with no subject within a detection tunnel of the PET scanner; obtaining second PET data relating to second background events occur during a second PET scan, the second PET scan being performed by the PET scanner with a subject within the detection tunnel; and generating, based on the first PET data and the second PET data, a target attenuation map of the subject, wherein the first background events and the second background events may occur when a first particle and a second particle are detected by crystal units of the PET scanner within a background coincident window, and the first particle and the second particle may be different types of particles produced by a radioactive decay of a crystal material of the PET scanner.

In some embodiments of the present disclosure, a target attenuation map of a subject may be generated by analyzing background events caused by radioactive decay of a crystal material of a detector of a PET scanner. The methods for attenuation map generation in the present disclosure may obviate the need of using a CT scanner or an external radiative source to scan the subject, thus the equipment requirements and the equipment cost may be reduced, the operation process may be simplified, and the radiation damage to the subject and other users (e.g., a doctor) may be reduced.

### BRIEF DESCRIPTION OF THE DRAWINGS

The present disclosure is further illustrated in terms of exemplary embodiments, and these exemplary embodiments are described in detail with reference to the drawings. These embodiments are not limited. In these embodiments, the same numeral indicates the same structure, wherein:

FIG. 1 is a schematic diagram illustrating an exemplary system for attenuation map generation according to some embodiments of the present disclosure;

FIG. 2 is a schematic diagram illustrating a radioactive decay process of Lu-176 according to some embodiments of the present disclosure;

FIG. 3 is a schematic diagram illustrating an exemplary process for determining a background event according to some embodiments of the present disclosure;

FIG. 4 is a flowchart illustrating an exemplary process for generating a target attenuation map according to some embodiments of the present disclosure;

FIG. 5 is a schematic diagram illustrating an exemplary process for generating a target attenuation map according to some embodiments of the present disclosure;

FIG. 6 is a schematic diagram illustrating an exemplary process for generating a PET image according to some embodiments of the present disclosure;

FIG. 7 is a block diagram illustrating an exemplary processing device according to some embodiments of the present disclosure; and

FIG. 8 is a schematic diagram illustrating an exemplary computing device according to some embodiments of the present disclosure.

### DETAILED DESCRIPTION

In order to illustrate the technical solutions relating to the embodiments of the present disclosure, a brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless stated otherwise or obvious from the context, the same reference numeral in the drawings refers to the same structure and operation.

It will be understood that the terms “system,” “device,” “unit,” and/or “module” used herein are one method to distinguish different components, elements, parts, sections, or assemblies of different levels in ascending order. However, the terms may be displaced by other expressions if they may achieve the same purpose.

As shown in the present disclosure and claims, unless the context clearly indicates exceptions, the words “a,” “an,” “one,” and/or “the” do not specifically refer to the singular, but may also include the plural. The terms “including” and “comprising” only suggest that the steps and elements that have been clearly identified are included, and these steps and elements do not constitute an exclusive list, and the method or device may also include other steps or elements.

The flowcharts used in the present disclosure may illustrate operations executed by the system according to embodiments in the present disclosure. It should be understood that a previous operation or a subsequent operation of the flowcharts may not be accurately implemented in order. Conversely, various operations may be performed in inverted order, or simultaneously. Moreover, other operations may be added to the flowcharts, and one or more operations may be removed from the flowcharts.

PET has been widely used in clinical examination and disease diagnosis. Normally, before a PET scan is performed by a PET scanner, a radioactive tracer (or referred to as a radioactive isotope) may be injected into a subject to be scanned. For example, the radioactive tracer may be generated by marking a material necessary for biological life metabolism (e.g., glucose, protein, nucleic acid, fatty acid, etc.) with a short-life radionuclide (e.g., 18F, 11C, etc.). One or more atoms of the radioactive tracer may be chemically incorporated into biologically active molecules in the subject. The active molecules may become concentrated in a tissue of interest within the subject. The radioactive tracer may undergo positron emission decay and emit positrons. A positron may travel a short distance (e.g., about 1 mm) within the tissue of interest, lose kinetic energy, and interact with an electron of the subject. The positron and the electron may annihilate and produce a pair of annihilation photons (e.g., annihilation photons having an energy level of 511 keV). The pair of annihilation photons (or radiation rays) may move in approximately opposite directions, and be detected by a detector of the PET scanner. PET data collected by the detector in the PET scan may be processed to reconstruct a PET image of the subject.

In the process of PET image reconstruction, physical correction, such as an attenuation correction and/or a scattering correction, may need to be performed on the PET data. The physical correction of the PET data may be performed based on an attenuation map of the subject. The attenuation map of the subject is generally obtained based on a computed tomography (CT) image of the subject. Alternatively, an external radioactive source (e.g., a rotating rod source, a rotating point source, etc.) may be used to irradiate the subject to collect image data, and the collected image data may be processed to obtain the attenuation map.

However, for a PET system without a CT scanner, such as a single PET system or a positron emission tomography-magnetic resonance (PET-MR) system, a CT image of the subject can not be acquired directly. For a PET system, such as a PET-CT system, the accuracy of the generated attenuation may be affected by the quality of the CT image. If the CT image has a poor quality, the generated attenuation map may also have a low accuracy, which may cause artifacts in the reconstructed PET image or inaccurate quantification based on the reconstructed PET image. A PET system without an external radioactive source is not able to obtain the attenuation map. Moreover, a CT scan or a scan performed by an external radioactive source may cause radiation damage to the subject or other users (e.g., a doctor) to a certain extent. At the same time, the price of the external radioactive source is relatively high, resulting in a high equipment cost.

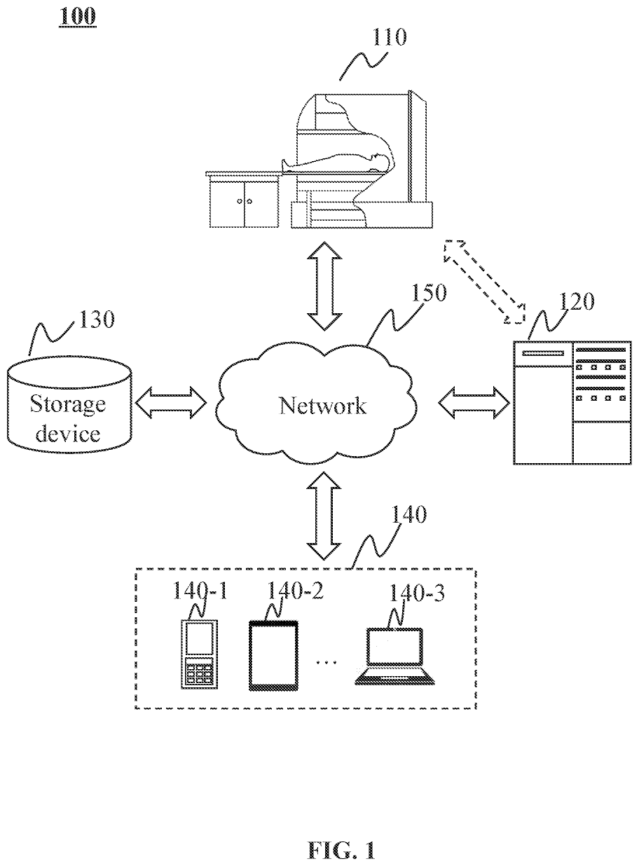

FIG. 1 is a schematic diagram illustrating an exemplary system 100 for attenuation map generation according to some embodiments of the present disclosure. The system 100 for attenuation map generation in the present disclosure may be referred to as the system 100 for short.

As shown in FIG. 1, in some embodiments, the system 100 may include a PET scanner 110, a processing device 120, a storage device 130, a terminal 140, and a network 150.

The PET scanner 110 may perform a PET scan on a subject. It should be noted that the PET scanner 110 is an exemplary scanner that performs an imaging or treatment operation on the subject using radionuclides. The PET scanner 110 may be replaced by any other scanning devices, such as a PET-CT scanner, a single-photon emission computed tomography (SPECT) scanner, a SPECT-CT scanner, a PET-MR scanner, or the like. For illustration purposes, the following descriptions are provided with reference to a PET scanner, and this is not intended to be limiting.

A detector of the PET scanner 110 may receive radiations from a radiation source (e.g., the subject) and measure the received radiation. In some embodiments, the PET scanner 110 may send data and information relating to the detector, such as an energy value of a gamma photon received by the detector, to the processing device 120. In some embodiments, the detector may include a plurality of crystal units, which may contain material that may undergo radioactive decay. The plurality of crystal units may detect particles produced by the radioactive decay. Based on these particles, data relating to background events (referred to as background event data for short) may be determined. Further, a target attenuation map of the subject may be generated based on the background event data. The target attenuation map may refer to an attenuation map of the subject corresponding to a target energy level (e.g., 511 keV).

In some embodiments, the PET scanner 110 may be a long-axis PET scanner or a short-axis PET scanner. The short-axis PET scanner may refer to a PET scanner whose length along an axial direction is less than a first predetermined threshold. The long-axis PET scanner may refer to a PET scanner whose length along the axial direction is greater than a second predetermined threshold. The second predetermined threshold may be the same or different from the first preset threshold. When the subject is scanned by the short-axis PET scanner, the subject may be moved several times to obtain PET data of different body parts. In some embodiments, the PET scanner 110 may be the long-axis PET scanner, such as a 2-meter PET scanner. Using the long-axis PET scanner, an image of the whole body of the subject may be acquired within a single scan without performing multiple scans, thus improving the efficiency of the PET scan.

In some embodiments, the PET scanner 110 may exchange data and/or information with other components in the system 100 (e.g., the processing device 120, the storage device 130, and the terminal 140) via the network 150. In some embodiments, the PET scanner 110 may be directly connected with other components in the system 100. In some embodiments, one or more components in system 100 (e.g., processing device 120, storage device 130) may be included within PET scanner 110.

The processing device 120 may process data and/or information obtained from other components of the system 100, and execute methods for attenuation map generation disclosed in the present disclosure. For example, the processing device 120 may generate the target attenuation map based on the background event data collected by the PET scanner 110. For another example, the processing device 120 may generate a medical image (e.g., the PET image) of the subject based on the target attenuation map and data relating to coincident events of the subject (named as coincident event data for short). In some embodiments, the processing device 120 may send the processed data, such as background event data, coincident event data, the target attenuation map, etc., to the storage device 130 for storage. In some embodiments, the processing device 120 may acquire data and/or information from the storage device 130, for example, background event data, coincident event data, various calculation formulas, etc., for executing the methods for attenuation map generation disclosed in the present disclosure.

In some embodiments, the processing device 120 may include one or more sub processing devices (e.g., a single core processing device or a multi-core and multi-chip processing device). Merely by way of example, the processing device 120 may include a central processing unit (CPU), an application specific integrated circuit (ASIC), an application specific instruction processor (ASIP), a graphics processing unit (GPU), a physics processing unit (PPU), a digital signal processor (DSP), a field programmable gate array (FPGA), a programmable logic circuit (PLD), a controller, a microcontroller unit, a reduced instruction set computer (RISC), a microprocessor, or the like, or any combination thereof.

The storage device 130 may store data or information relating to the system 100. In some embodiments, the storage device 130 may store data and/or information collected by the PET scanner 110, such as background event data, coincident event data, or the like. In some embodiments, the storage device 130 may store data processed or generated by the processing device 120, for example, the target attenuation map of the subject, or the like. The storage device 130 may include one or more storage components, and each storage component may be an independent device or a portion of another device. The storage device 130 may be local or implemented based on a cloud.

The terminal 140 may enable user interactions between a user and the system 100. For example, the user may input an operation instruction via the terminal 140 to control the PET scanner 110 to implement a specific operation, for example, irradiating and imaging a specific body part of the subject. In some embodiments, the terminal 140 may instruct the processing device 120 to execute the methods for attenuation map generation disclosed in the present disclosure. In some embodiments, the terminal 140 may receive a reconstructed image from the processing device 120, so that the user may accurately judge a condition of the subject to perform an effective and targeted examination and/or treatment for the subject. In some embodiments, the terminal 140 may include a mobile device 140-1, a tablet computer 140-2, a laptop computer 140-3, a desktop computer, or other devices with functions of input and/or output, or any combination thereof.

The network 150 may connect various components in the system 100 and/or connect the system 100 with an external device. In some embodiments, one or more components in system 100 (e.g., the PET scanner 110, the processing device 120, the storage device 130, the terminal 140) may send data and/or information to other components via network 150. In some embodiments, the network 150 may include a wired network and/or a wireless network.

It should be noted that the above descriptions are provided for illustration purpose only, and is not intended to limit the scope of the present disclosure. For those skilled in the art, various changes and modifications may be made under the guidance of the contents of the present disclosure. The features, structures, methods, and other features of the exemplary embodiments described in the present disclosure may be combined in various ways to obtain additional and/or alternative exemplary embodiments. For example, the processing device 120 may be based on a cloud platform, such as a public cloud, a private cloud, a community cloud, a hybrid cloud, or the like. However, these changes and modifications do not deviate from the scope of the present disclosure.

As mentioned above, a plurality of crystal units of a PET detector may include material that may undergo radioactive decay. Background event data may be determined based on the radioactive decay of the crystal material, and a target attenuation map of the subject may be generated based on the background event data. As used herein, when the crystal units of the PET scanner detect a first particle and a second particle generated by the radioactive decay of the crystal material within a background coincident window, it may be determined that a background event occurs. The first particle and the second particle may be different types or the same type of particles.

Merely by way of example, the plurality of crystal units of the PET detector may include a sodium iodide (Nal) crystal unit, a bismuth germanate (BGO) crystal unit, a lutetium silicate (LSO) crystal unit, an yttrium lutetium silicate (LYSO) crystal unit, or the like. The LSO crystal unit and the LYSO crystal unit may include Lu-176, which undergoes radioactive decay. Lu-176 releases particles in the radioactive decay.

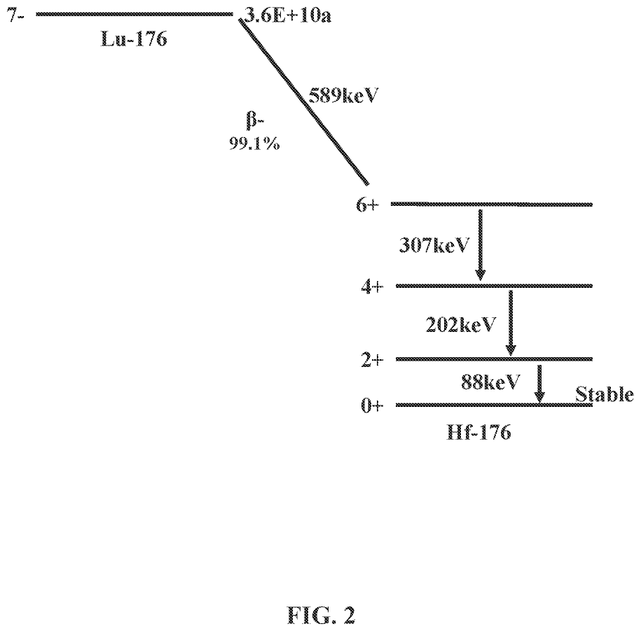

FIG. 2 is a schematic diagram illustrating an exemplary radioactive decay process of Lu-176. As shown in FIG. 2, the radioactive decay of Lu-176 may include a beta decay and a cascade gamma decay. The beta decay may generate a beta electron with an energy in a range of 0 keV˜ 589 keV. The gamma decay may generate three kinds of gamma photons, one having the energy of 307 keV, one having the energy of 202 keV, and the other one having the energy of 88 keV. If the radioactive decay of Lu-176 occurs in a crystal unit, the beta electron may be detected in the crystal unit, and one or more of the gamma photons may escape from the crystal unit and be detected by other crystal unit(s).

Based on the simultaneity and different energies of the beta decay and the gamma decay, a background event caused by the radioactive decay of Lu-176 may be identified. For example, if a beta electron (i.e., a first particle) and a gamma photon (i.e., the second particle) produced by the radioactive decay of Lu-176 are detected by different crystal units in the background coincident window, it may be determined that a background event occurs.

The background coincident window may relate to an energy and/or a detection time of a particle, and may be used to identify particles produced by the same radioactive decay. In some embodiments, the background coincident window may include at least one energy window and at least one time window. An energy window may relate to an energy of a particle, and a time window may relate to a detection time difference between different particles. For example, the background coincident window may include a first energy window corresponding to the first particle, a second energy window corresponding to the second particle, and a time window. The first energy window may be an energy range of the first particle, the second energy window may be an energy range of the second particle, and the time window may be a range of a detection time difference between the first particle and the second particle.

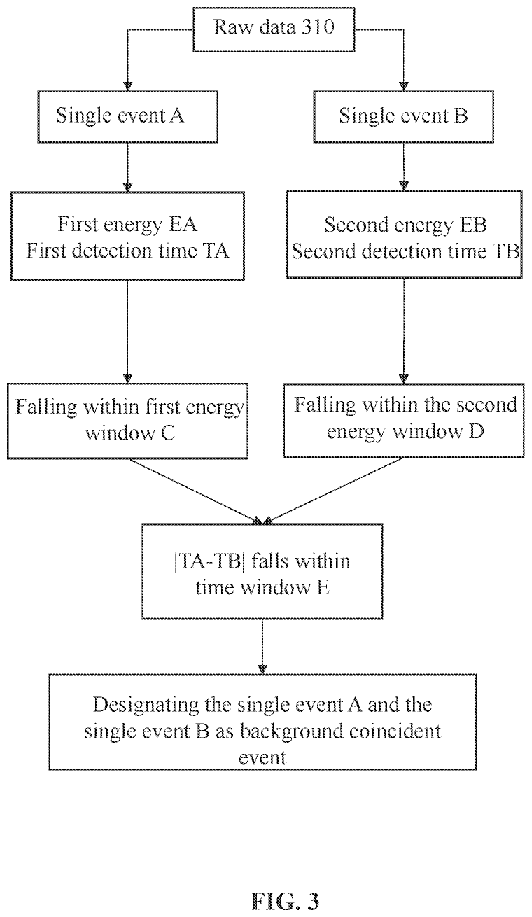

For example, FIG. 3 is a schematic diagram illustrating an exemplary process for determining a background event according to some embodiments of the present disclosure. In some embodiments, process 300 as shown in FIG. 3 may be executed by the processing device 120 and/or a processor of the PET scanner 110.

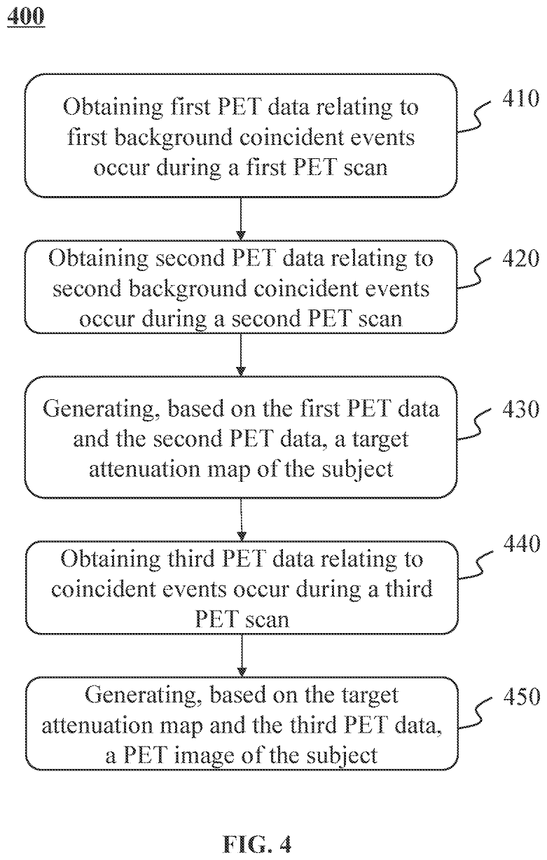

As shown in FIG. 3, a single event A and a single event B may be determined based on raw data 310. The raw data 310 may be acquired by a PET scanner during a first PET scan (e.g., a blank PET scan) or a second PET scan (e.g., a PET scan performed on a subject without being injected with any radioactive tracer) described in FIG. 4. The single event A may refer to that a crystal unit of the PET scanner receives a first particle P1, and the single event B may refer to that another crystal unit of the PET scanner receives a second particle P2. As used herein, the single event A may occur prior to the single event B. Based on the single event A, a first energy EA and a first detection time TA of the first particle P1 may be determined. Based on the single event B, a second energy EB and a second detection time TB of the second particle P2 may be determined. Further, the background coincident window, including a first energy window C, a second energy window D, and a time window E, may be obtained.

Further, whether the first particle P1 and the second particle P2 meet a predetermined condition may be determined based on the first energy EA of the first particle P1, the first detection time TA of the first particle P1, the second energy EB of the second particle P2, the second detection time TB of the second particle P2, and the background coincident window. In response to determining that the first particle P1 and the second particle P2 meet the predetermined condition, it may be determined that a background event occurs. In response to determining that the first particle P1 and the second particle P2 do not meet the predetermined condition, it may be determined that no background event occurs.

For example, as shown in FIG. 3, if the first energy EA falls within the first energy window C, the second energy EB falls within the second energy window D, and |TA-TB| falls within the time window E, the first particle P1 and the second particle P2 may satisfy the predetermined condition, and the single event A and the single event B may be designated as a background event.

For example, it is assumed that the first particle P1 is a beta electron produced by the radioactive decay of Lu-176, the second particle P2 is a gamma photon produced by the radioactive decay of Lu-176. In such cases, the first energy window C may be [0, E_{βmax}+3σ]. In some embodiments, the first energy window may be [LLD, E_{βmax}+3σ]. LLD refers to the lowest energy threshold relating to a deposited energy E_{β} of the beta electron in a crystal unit, and E_{βmax} refers to the maximum value of the deposited energy E_{β} of the beta electron in the crystal unit. In some embodiments, LLD may be 50 keV, 88 keV, 100 keV, or the like. E_{βmax} may be 589 keV, or the like. The second energy window D may be [E_{γ}−3σ, E_{γ}+3σ]. E_{γ} refers to a deposited energy of the gamma photon in a crystal unit. E_{γ} may be 88 keV, 202 keV, 307 keV, or the like. σ may be a standard deviation of the Gaussian energy distribution.

Merely by way of example, the first energy window C may be [100 keV, 589 keV+3σ]. The second energy window D may be one of [307 keV−3σ, 307 keV+3σ], [202 keV−3σ, 202 keV+3σ], and [88 keV−3a, 88 keV+3σ]. In some embodiments, determining the first energy window based on the LLD may reduce data noise. In some embodiments, a plurality of second energy windows D may be provided to determine background events corresponding to different energy levels. For example, a background event corresponding to an energy level of 307 keV may be determined based on the second energy window [307 keV−3σ, 307 keV+3σ]. A background event corresponding to an energy level of 202 keV may be determined based on a second energy window [202 keV−3σ, 202 keV+3σ]. A background event corresponding to an energy level of 88 keV may be determined based on the second energy window [88 keV−3σ, 88 keV+3σ].

In some embodiments, the time window E may be [0, ΔTₘₐₓ+3σ′], where ΔTₘₐₓ is a maximum absolute value of the detection time difference between the beta electron and the gamma photon. σ′ is standard deviation of the Gaussian time distribution. ΔTₘₐₓ may be determined based on a propagation speed of the gamma photon, a position of a crystal unit that detects the beta electron, and a position of a crystal unit that detects the gamma photon.

In some embodiments, a background event may be assigned to a line of response (LOR) joining the two relevant crystal units that detect the background event. Since a signal intensity of the background event is probably weak, in order to increase a count of background events received on a LOR and reduce the statistical noise, two or more original LORs corresponding to the same pair of crystal units may be merged into a combined LOR.

It should be noted that the descriptions of FIG. 2 and FIG. 3 are merely for illustration purpose. For those skilled in the art, various changes and modifications may be made under the guidance of the contents of the present disclosure. For example, a background event may be determined based on the radioactive decay of any other crystal materials. For another example, the background coincident window used to determine the background event may be different from the examples provided above.

FIG. 4 is a flowchart illustrating an exemplary process of generating a target attenuation map according to some embodiments of the present disclosure. In some embodiments, process 400 in FIG. 4 may be executed by the system 100. For example, the process 400 may be implemented as a set of instructions (e.g., an application) stored in a storage device (e.g., the storage device 130) of the system 100, and be invoked and/or executed by the processing device 120.

In 410, the processing device 120 may obtain first PET data relating to first background events occur during a first PET scan. In some embodiments, operation 410 may be performed by a data acquisition module 710.

The first PET scan may be performed by a PET scanner with no subject within a detection tunnel of the PET scanner. That is, the first PET scan may be performed to scan the air. The first PET scan may also be referred as a blank PET scan.

The first PET data may relate to the first background events occur during the first PET scan. As mentioned above, background events may be caused by a radioactive decay of crystal material of the PET scanner. For example, during the first PET scan, a first particle P1 and a second particle P2 produced by a radioactive decay of the crystal material may be detected by different crystal units in a background coincident window, which may be considered as a first background event.

In some embodiments, the first PET data may include various information relating to the first background events, such as a first energy of the first particle P1, a first detection time of the first particle P1, a second energy of the second particle P2, a second detection time of the second particle P2, a position of a crystal unit detecting the first particle P1, a position of a crystal unit detecting the second particle P2, or the like, or any combination thereof. In some embodiments, the first PET data may include list mode data, which includes a list of crystal unit information, detection time information, and energy information of each first background event. In some embodiments, the first PET data may include data of background events received by at least one original LOR or combined LOR (e.g., a LOR combined by original LORs).

In some embodiments, the first background events may be determined by a computing device (e.g., the processing device 120 or a processor of the PET scanner) based on first raw data (e.g., list-mode data) collected by the PET scanner in the first PET scan. The process 300 as described in connection with FIG. 3 may be performed on the first raw data to determine the first background events. Merely by way of example, the first raw data may include data relating to the first particle P1 and the second particle P2 detected by the crystal units of the PET scanner in the first PET scan. The processing device 120 may obtain the background coincident window that includes a first energy window corresponding to the first particle P1 (e.g., the first energy window C as described in FIG. 3), a second energy window corresponding to the second particle P2 (e.g., the second energy window D as described in FIG. 3), and a time window (e.g., the time window E as described in FIG. 3). The processing device 120 may further determine whether the first particle P1 and the second particle P2 meet a predetermined condition based on the first energy of the first particle P1, the first detection time of the first particle P1, the second energy of the second particle P2, the second detection time of the second particle P2, and the background coincident window. If the first particle P1 and the second particle P2 meet the predetermined condition, the processing device 120 may determine the detections of the first particle P1 and the second particle P2 as a first background event occurred in the first PET scan. In some embodiments, the first PET data may be previously determined and stored in a storage device (e.g., the storage device 130), and the processing device 120 may obtain the first PET data from the storage device.

In some embodiments, when the second particle P2 is a gamma photon released in the radioactive decay of the Lu-176, the second energy of the second particle P2 may be one of 88 keV, 202 keV, and 307 keV. In some embodiments, considering that the count of photons corresponding to a certain energy level may be relatively low, in order to improve the accuracy of the target attenuation map, a plurality of groups of first PET data corresponding to a plurality of energy levels may be generated. For example, the second energy window may be set as [307 keV−3σ, 307 keV+3σ] to determine a group of first PET data corresponding to an energy level of 307 keV. For another example, the second energy window may be set as [202 keV−3σ, 202 keV+3σ] to determine another group of first PET data corresponding to an energy level of 202 keV.

In 420, the processing device 120 may obtain second PET data relating to second background events occur during a second PET scan. In some embodiments, operation 420 may be performed by the data acquisition module 710.

The second PET scan may be performed by the PET scanner with a subject within the detection tunnel, and the subject may have not been injected with any radioactive tracer. In some embodiments, the subject may include a living body (e.g., a patient), a phantom, or the like. The second PET data may relate to the second background events occur during the second PET scan. For example, during the second PET scan, a first particle P1′ and a second particle P2′ produced by a radioactive decay of the crystal material may be detected by different crystal units in the background coincident window, which may be considered as a second background event. The determination of the second background events is similar to the determination of the first background events, and the acquisition of the second PET data is similar to the acquisition of the first PET data, which may not be described herein.

In some embodiments, during the second PET scan, the subject (e.g., a patient) may be placed in the PET scanner's field of view, and background event data of the subject may be acquired as the second PET data.

In 430, the processing device 120 may generate, based on the first PET data and the second PET data, a target attenuation map of the subject. In some embodiments, operation 430 may be performed by an attenuation map generation module 720.

The target attenuation map may reflect an attenuation ability of the subject with respect to radiations (e.g., gamma rays, X rays) with a second energy level (i.e., a target energy level). For example, the target attenuation map may include attenuation coefficients of each point on the subject with respect to gamma rays with an energy of 511 keV.

In some embodiments, the first PET data and the second PET data corresponding to each crystal unit of the PET scanner may be obtained by analyzing the first PET data and the second PET data. Further, based on the first PET data and the second PET data corresponding to each crystal unit, the target attenuation image may be obtained. For a pair of crystal units, when a particle produced by a background event passes through the subject along a connecting line between the pair of crystal units, a count of the particles may be reduced due to an attenuation effect of the subject. An attenuation degree of the subject along the connecting line may be determined based on a degree of the count reduction of the particles. In some embodiments, the target attenuation map may be obtained based on attenuation degrees corresponding to all pairs of crystal units.

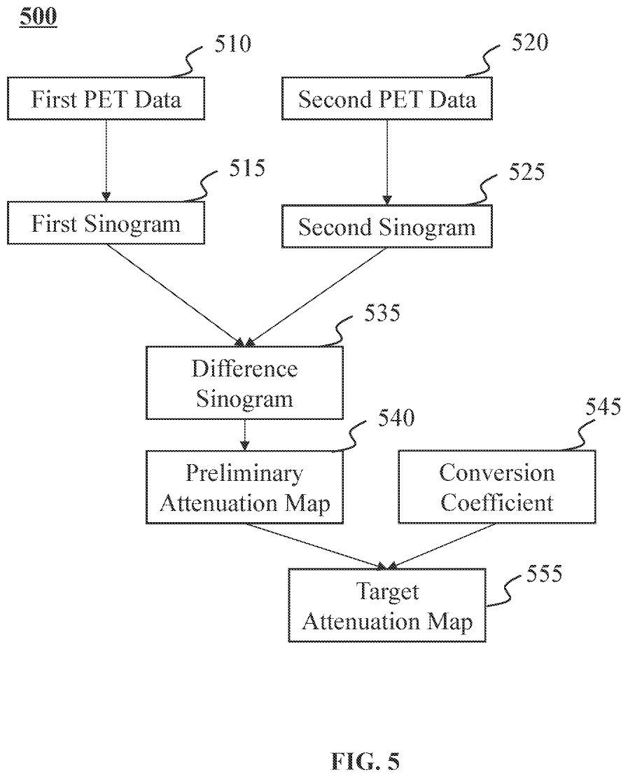

In some embodiments, the processing device 120 may generate a preliminary attenuation map based on the first PET data and the second PET data. The preliminary attenuation map may be an attenuation map of the subject with respect to gamma rays with a first energy level, and reflect an attenuation ability of the subject with respect to gamma rays with the first energy level. Further, the processing device 120 may obtain the target attenuation map based on the preliminary attenuation map and a conversion coefficient. The first energy level and the second energy level may be any two different energy levels. For example, the first energy level may include at least one of 88 keV, 202 keV, 307 keV, and the second energy level may include at least one of 511 keV, 662 keV, or the like. More descriptions regarding the generation of the target attenuation map based on the first PET data and the second PET data may be found elsewhere in the present disclosure. See, e.g., FIG. 5 and relevant descriptions thereof.

In 440, the processing device 120 may obtain third PET data relating to coincident events occur during a third PET scan. In some embodiments, operation 440 may be performed by the data acquisition module 710.

The third PET scan may be performed by the PET scanner on the subject who has been injected with a radioactive tracer. For example, the radioactive tracer may be injected into the subject (e.g., a patient) before the third PET scan. During the third PET scan, a positron generated by the decay of the radioactive tracer and an electron in the subject may annihilate and produce a pair of gamma photons (e.g., gamma photons with an energy level of 511 keV) with opposite propagation directions. Original PET data may be collected by the detector of the PET scanner by detecting the pair of gamma photons. Based on the original PET data, the third PET data relating to the coincident events occur during the third PET scan may be determined. For example, if two gamma photons are received and interact with two scintillator units within a certain coincidence time window (e.g., 1 nanosecond, 2 nanoseconds, 5 nanoseconds, 10 nanoseconds, 20 nanoseconds, etc.), the two photons may be deemed to come from the same annihilation, and regarded as a coincidence event.

In 450, the processing device 120 may generate, based on the target attenuation map and the third PET data, a PET image of the subject. In some embodiments, operation 450 may be performed by a PET image generation module 730.

In some embodiments, the third PET data may be corrected based on the target attenuation map, and then the PET image may be reconstructed based on corrected third PET data.

In some embodiments of the present disclosure, the target attenuation map of the subject may be generated based on the first PET data and the second PET data relating to the background events. The third PET data may be corrected based on the target attenuation map to improve the accuracy of the PET image reconstructed based on the corrected third PET data. The methods for attenuation map generation disclosed in the present disclosure may be performed without performing any additional scan of the subject using a CT device or additional radiation sources, thus the radiation damage to the subject and the equipment cost may be reduce, and the efficiency of PET scanning may be improved.

FIG. 5 is a schematic diagram illustrating an exemplary process for generating a target attenuation map according to some embodiments of the present disclosure. In some embodiments, one or more operations of process 500 as shown in FIG. 5 may be performed to achieve at least part of operation 430 as described in connection with FIG. 4.

As shown in FIG. 5, the processing device 120 may obtain first PET data 510 and second PET data 520. The processing device 120 may generate a first sinogram 515 based on the first PET data 510, and generate a second sinogram 525 based on the second PET data 520. The processing device may generate a difference sinogram 535 between the first sinogram 515 and the second sinogram 525. For example, the difference sinogram 535 may be determined by dividing the first sinogram 515 by the second sinogram 525 or dividing the second sinogram 525 by the first sinogram 515. Further, the difference sinogram 535 may be reconstructed to generate a preliminary attenuation map 540.

In some embodiments, the difference sinogram 535 and the preliminary attenuation map 540 may correspond to a first energy level. For example, since the first background events are determined based on a deposited energy E_{γ} of gamma photons, the difference sinogram 535 and the preliminary attenuation map 540 may reflect an attenuation degree of the gamma photons with the energy of E_{γ} produced by the first background events after passing through the subject. The energy of a gamma photon released during the radiation decay of Lu-176 may be one of 88 keV, 202 keV, or 307 keV. That is, E_{γ} may be one of 88 keV, 202 keV, or 307 keV. Therefore, in some embodiments, the first energy level may be one of 88 keV, 202 keV, or 307 keV.

In some embodiments, when the count of gamma photons corresponding to a certain energy level is small (e.g., smaller than a threshold), an attenuation map corresponding to the certain energy level may have a low accuracy. In order to improve the accuracy of the target attenuation map, a plurality of preliminary attenuation maps corresponding to a plurality of first energy levels may be determined, and the plurality of preliminary attenuation maps may be fused to determine a final preliminary attenuation map. For example, the first energy level may include two or more of 88 keV, 202 keV, or 307 keV. A preliminary attenuation map corresponding to each energy level may be generated, and these preliminary attenuation maps corresponding to different energy levels may be averaged to determine the final preliminary attenuation map. A preliminary attenuation map corresponding to a certain energy level may be generated based on the first PET data and the second PET data corresponding to the certain energy level.

After the preliminary attenuation map 540 is generated, the processing device 120 may generate a target attenuation map 555 based on the preliminary attenuation map 540 and a conversion coefficient 545. The target attenuation map 555 may be an attenuation map of the subject with respect to the gamma rays with a second energy level. In some embodiments, the second energy level may include one of 511 keV, 662 keV, or the like. In some embodiments, the preliminary attenuation map 540 may be designated as μ′. The target attenuation map 555 may be designated as μ. The target attenuation map may be obtained by formula (1) as below:

$$μ = k {μ}^{′} , \qquad ( 1 )$$

where k represents the conversion coefficient.

In some embodiments, the conversion coefficient 545 may reflect a difference between an attenuation ability of the subject with respect to the gamma rays of the first energy level and an attenuation ability of the subject with respect to the gamma rays of the second energy level. In some embodiments, the conversion coefficient 545 may be a constant, which may be determined manually or based on data analysis. For example, the processing device 120 may acquire a first attenuation coefficient of a reference material with respect to gamma rays with the first energy level, and a second attenuation coefficient of the reference material with respect to gamma rays with the second energy level. The processing device 120 may further determine the conversion coefficient 545 based on the first attenuation coefficient and the second attenuation coefficient.

In some embodiments, the reference material may be predetermined specific material, such as water. In some embodiments, the conversion coefficient k may be obtained by determining a first attenuation coefficient (i.e., μ′_{H2O}) of water with respect to gamma rays with the first energy level, and a second attenuation coefficient (i.e., μ′_{H2O}) of water with respect to gamma rays with the second energy level. The conversion coefficient k may be obtained based on formula (2) as below:

$$k = \frac{{μ}_{H 2 O}}{{μ}_{H 2 O}^{′}} \qquad ( 2 )$$

Since the ratios (i.e., a conversion coefficient) of the first attenuation coefficients and the second attenuation coefficients of different materials may be the same or substantially the same, the conversion coefficient of the subject may be determined based on the first attenuation coefficient and the second attenuation coefficient of the reference material. By determining the conversion coefficient based on the reference material, additional measurement on the subject may be omitted, and the conversion coefficient may be used to generate target attenuation maps of different subjects, thus improving the efficiency of PET scanning.

In some embodiments, the processing device 120 may perform noise reduction operation on the first PET data 510 and the second PET data 520 before generating the target attenuation map 555. The noise reduction operation may include at least one of spatial domain filtering, transform domain filtering, or morphology noise filtering. The spatial filtering may refer to that when a plurality of signals superposed together in the time domain occupy the same frequency band, signal spatial processing may be performed based on a spatial separation of signals from different directions. The transform domain filtering may refer to transforming signals from the time domain to the frequency domain, and performing filtering based on frequency domain signals. The morphology noise filtering may refer to opening noise data to remove background noise, and then closing the noise data to remove data noise. By reducing the noise of the first PET data 510 and the second PET data 520, the effect of data noise may be reduced, and the accuracy of the target attenuation image 555 may be further improved.

In some embodiments, the processing device 120 may perform a first noise reduction operation on the first PET data 510 to generate first target PET data. The processing device 120 may perform a second noise reduction operation on the second PET data 520 to generate second target PET data. The processing device 120 may generate the target attenuation map 555 of the subject based on the first target PET data and the second target PET data. For example, the processing device 120 may generate the preliminary attenuation map 540 described above based on the first target PET data and the second target PET data. The processing device 120 may further generate the target attenuation map 555 based on the preliminary attenuation map.

In some embodiments, at least one of the first noise reduction operation and the second noise reduction operation may be performed based on a noise reduction model. The noise reduction model may include a trained machine learning model, such as a neural network model or algorithms including a local smoothing filter, a gaussian smoothing model, a neighborhood filter, a frequency domain filter, a wavelet thresholding model, or a non-local means algorithm, or the like. An input of the noise reduction model may include PET data, and an output of the noise reduction model may include denoised PET data. In some embodiments, the first noise reduction operation on the first PET data 510 may include inputting the first sinogram 515 into the noise reduction model to obtain the first target PET data after noise reduction. In some embodiments, the second noise reduction operation on the second PET data 520 may include inputting the second sinogram 525 into the noise reduction model to obtain the second target PET data after noise reduction.

In some embodiments, the noise reduction model may be generated based on a plurality of training samples. Each training sample may include first sample PET data corresponding to a first acquisition duration and second sample PET data corresponding to a second acquisition duration. The first acquisition duration and the second acquisition duration may be different. For example, the first acquisition duration may be a short acquisition duration shorter than a duration threshold (e.g., the first acquisition duration may be 1-5 minutes), and a noise level of the corresponding first sample PET data noise may be high. The second acquisition duration may be a long acquisition duration longer than the duration threshold (e.g., the second acquisition duration may be 20-30 minutes), and a noise level of the corresponding second sample PET data may be low. In some embodiments, two PET scans with different acquisition durations may be performed under the same scanning condition to obtain the first sample PET data and the second sample PET data. For example, two blank PET scans with different acquisition durations may be performed to acquire the first sample PET data and the second sample PET data. As another example, two PET scans with different acquisition durations may be performed on a same sample subject injected with radioactive tracer or without being injected with radioactive tracer, so as to acquire the first sample PET data and the second sample PET data. By performing PET scans with different acquisition durations, data with different noise levels may be obtained as training data to train the noise reduction model.

In some embodiments, the noise reduction model may be constructed by using a framework of TensorFlow. In some embodiments, a preliminary neural network model may be constructed based on the framework of TensorFlow. Then a large amount of historical PET data may be obtained as training samples to train the preliminary neural network model. Finally, a trained neural network model may be obtained. In some embodiments, the noise reduction model may be generated in advance by the processing device 120 or other processing devices (e.g., the processing device of a supplier of the noise reduction model) and stored in the storage device. The processing device 120 may acquire the noise reduction model from the storage device and apply it to the noise reduction operations on the first PET data and the second PET data.

In some embodiments, the noise reduction model may also be used to enhance information of the first PET data and the second PET data. For example, the noise reduction model may increase an amount of the first PET data, or establish a correlation between data of different first background events, to improve the accuracy of the first PET data relating to the first background events.

In some embodiments of the present disclosure, the first target PET data and the second target PET data after noise reduction may be obtained by inputting the first PET data and the second PET data into the trained noise reduction model. The noise reduction model may have advantages including having a strong computing ability, a strong adaptability, and a high robustness. The noise reduction model may simultaneously process a large amount of data relating to background events, thus improving the speed and effect of noise reduction.

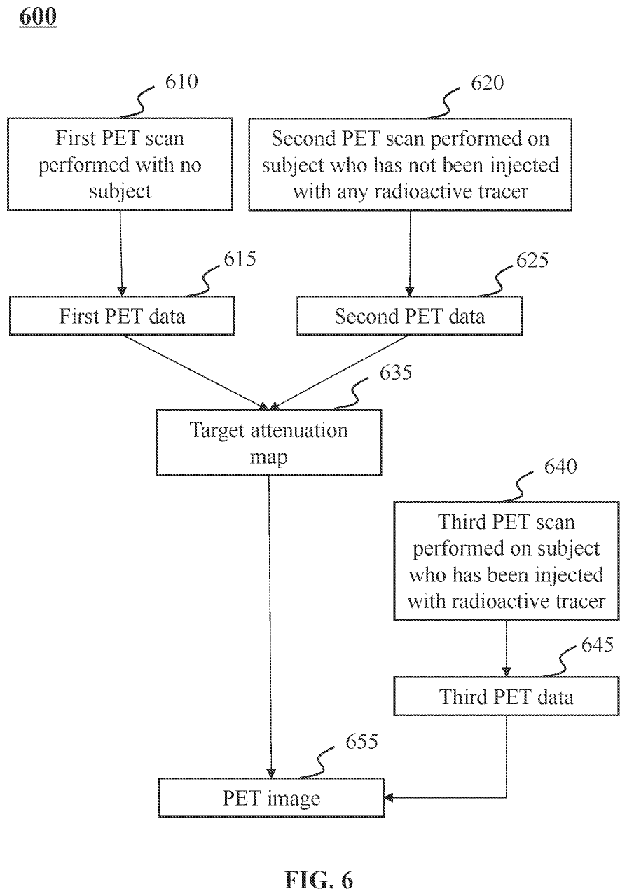

FIG. 6 is a schematic diagram illustrating an exemplary process for generating a PET image according to some embodiments of the present disclosure. Process 600 shown in the FIG. 6 may be exemplary embodiments of the process 400 shown in FIG. 4.

As shown in FIG. 6, in 610, a first PET scan may be performed by a PET scanner with no subject within a detection tunnel of the PET scanner, then first PET data 615 may be obtained. In 620, a second PET scan may be performed by the PET scanner with a subject within a detection tunnel of the PET scanner, then second PET data 625 may be obtained. The first PET data 615 and the second PET data 625 may be background event data. More description of obtaining the background event data may be found elsewhere in the present disclosure. See, e.g., operations 410, 420, and relevant descriptions thereof, which may not be described herein.

Further, the processing device 120 may generate a target attenuation map 635 of the subject based on the first PET data 615 and the second PET data 625. The target attenuation map 635 may be an attenuation map of the subject with respect to the gamma rays with a second energy level. In some embodiments, the processing device 120 may generate a preliminary attenuation map of the subject with respect to the gamma rays with a first energy level based on the first PET data 615 and the second PET data 625, and obtain the target attenuation map 635 based on the preliminary attenuation map and a conversion coefficient. More descriptions of generating the target attenuation map of the subject may be found elsewhere of the present disclosure, for example, operation 430 and relevant descriptions thereof.

In 640, a third PET scan may be performed by the PET scanner on the subject who has been injected with a radioactive tracer, and third PET data 645 may be obtained. The third PET data 645 may be coincident event data. More descriptions of obtaining coincident event data may be found elsewhere in the present disclosure, for example, operation 440 and relevant descriptions thereof.

After obtaining the target attenuation map 635 and the third PET data 645, the processing device 120 may generate a PET image 655 of the subject based on the target attenuation map 635 and the third PET data 645. More descriptions of generating a PET image based on a target attenuation map and third PET data may be found elsewhere in the present disclosure, for example, operation 450 and relevant descriptions thereof, which may not be described herein.

It should be noted that the above descriptions of the process 400 to the process 600 is merely for illustration purpose, and do not limit the scope of the present disclosure. For those skilled in the art, various modifications and changes may be made to the process 400 to the process 600 under the guidance of the present disclosure. However, these modifications and changes are still within the scope of the present disclosure. For example, operation 440 may be performed before operations 410 and 420. Alternatively, operations 410, 420, and 440 may be performed simultaneously.

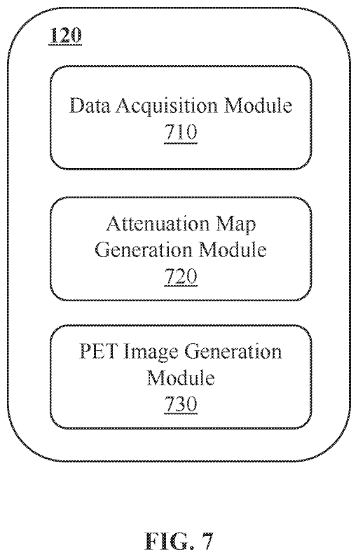

FIG. 7 is a block diagram illustrating an exemplary processing device 120 according to some embodiments of the present disclosure.

As shown in FIG. 7, the processing device 120 may include a data acquisition module 710, an attenuation map generation module 720, and a PET image generation module 730.

In some embodiments, the data acquisition module 710 may be configured to obtain first PET data relating to first background events occur during a first PET scan, and the first PET scan may be performed by a PET scanner with no subject within a detection tunnel of the PET scanner. The data acquisition module 710 may also obtain second PET data relating to second background events occur during a second PET scan, and the second PET scan may be performed by the PET scanner with a subject within the detection tunnel. The data acquisition module 710 may also be configured to obtain third PET data relating to coincident events occur during a third PET scan, the third PET scan may be performed by the PET scanner with the subject being injected a radioactive tracer.

In some embodiments, the attenuation map generation module 720 may be configured to generate a target attenuation map of the subject based on the first PET data and the second PET data. The first background events and the second background events may occur when a first particle and a second particle are detected by crystal units of the PET scanner within a background coincident window, and the first particle and the second particle may be different types of particles produced by a radioactive decay of a crystal material of the PET scanner.

In some embodiments, the PET image generation module 730 may be configured to generate a PET image of the subject based on the target attenuation map and the third PET data.

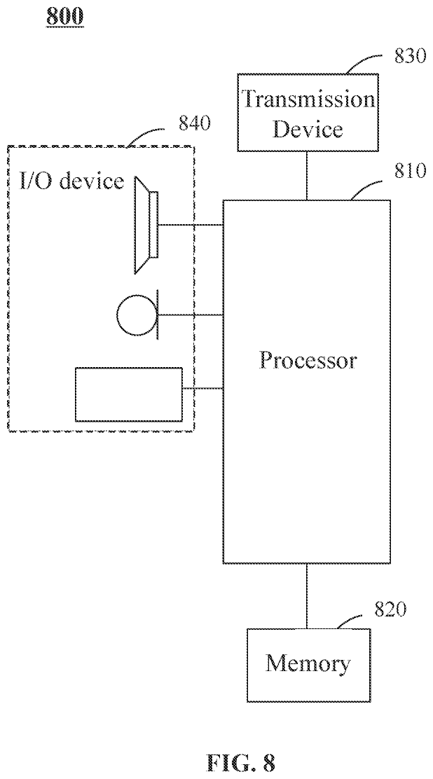

FIG. 8 is a schematic diagram illustrating an exemplary computing device 800 according to some embodiments of the present disclosure. In some embodiments, one or more components in system 100 may be implemented by computing device 800. For example, the processing device 120 may be implemented by the computing device 800.

As shown in FIG. 8, the computing device 800 may include a processor 810, a memory 820, a transmission device 830, and an input/output (I/O) device 840.

The processor 810 may be configured to execute instructions. In some embodiments, the processor 810 may execute the instructions stored in the memory 820, so that the computing device 800 may perform various applications and data processing operations, for example, performing the methods for attenuation map generation shown in the process 400 and the process 600.

The memory 820 may be configured to store computer programs and data. In some embodiments, the memory 820 may store instructions corresponding to the methods for attenuation map generation disclosed in the present disclosure, for example, instructions corresponding to the process 400 and the process 600.

The transmission device 830 may be configured to receive or transmit data. In some embodiments, the transmission device 830 may transmit data via a network (e.g., the network 150). In some embodiments, the transmission device 830 may include a network interface controller (NIC) for connecting with other network devices. In some embodiments, the transmission device 830 may include a radio frequency (RF) module for wireless communication with the Internet.

The I/O device 840 may be configured to interact with a user. In some embodiments, the I/O device 840 may include any device that may receive user input and/or may output data to the user, such as a keyboard, a mouse, a handwriting device, a display, a printer, a touch screen, or the like. In some embodiments, the I/O device 840 may be used for a user to input an instruction (e.g., an instruction to execute the methods for attenuation map generation disclosed in the present disclosure), to drive the processor 810 to execute the instruction.

The possible beneficial effects of the embodiments of the present disclosure may include, but be not limited to: (1) The target attenuation map of the subject may be generated based on background event data, and the coincident event data may be corrected based on the target attenuation map to generate a PET image of the subject. The methods for attenuation map generation may obtain the target attenuation map without a CT scanner, an external radiative source, or an installation device, thus the equipment requirements and the equipment cost may be greatly reduced, the operation process may be simplified, and the radiation damage to the subject and other users (e.g., a doctor) may be reduce; (2) A preliminary attenuation map of the subject with respect to gamma rays with a first energy level may be determined based on first PET data and second PET data, and the preliminary attenuation map may be converted to a target attenuation map corresponding to a target energy level (i.e., the second energy level) by a conversion coefficient. The conversion coefficient may be determined based on attenuation coefficients of the reference material. The conversion coefficient may be obtained without performing an additional PET scan on the subject, thus the efficiency of PET scanning may be improved. (3) By using the noise reduction model to perform the noise reduction operation on the first PET data and the second PET data, the efficiency and accuracy of noise reduction may be improved, and then the accuracy of the target attenuation map and the PET image may be improved. It should be noted that different embodiments may produce different beneficial effects. In different embodiments, the possible beneficial effects may be any combination of the above, or any other possible beneficial effects.

Having thus described the basic concepts, it may be rather apparent to those skilled in the art after reading this detailed disclosure that the foregoing detailed disclosure is intended to be presented Merely by way of example and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Meanwhile, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution—e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Contents of each of patents, patent applications, publications of patent applications, and other materials, such as articles, books, specifications, publications, documents, etc., referenced herein are hereby incorporated by reference, excepting any prosecution file history that is inconsistent with or in conflict with the present document, or any file (now or later associated with the present disclosure) that may have a limiting effect to the broadest scope of the claims. It should be noted that if the description, definition, and/or terms used in the appended materials of the present disclosure is inconsistent or conflicts with the content described in the present disclosure, the use of the description, definition and/or terms of the present disclosure shall prevail.

Finally, it should be understood that the embodiments described in the present disclosure merely illustrates the principles of the embodiments of the present disclosure. Other modifications may be within the scope of the present disclosure. Accordingly, by way of example, and not limitation, alternative configurations of embodiments of the present disclosure may be considered to be consistent with the teachings of the present disclosure. Accordingly, the embodiments of the present disclosure are not limited to the embodiments explicitly introduced and described by the present disclosure.

1. A method for attenuation map generation, comprising:
obtaining first positron emission tomography (PET) data relating to first background events occur during a first PET scan, the first PET scan being performed by a PET scanner with no subject within a detection tunnel of the PET scanner;
obtaining second PET data relating to second background events occur during a second PET scan, the second PET scan being performed by the PET scanner with a subject within the detection tunnel; and
generating, based on the first PET data and the second PET data, a target attenuation map of the subject, wherein the first background events and the second background events occur when a first particle and a second particle are detected by crystal units of the PET scanner within a background coincident window, and the first particle and the second particle are different types of particles produced by a radioactive decay of a crystal material of the PET scanner.

2. The method of claim 1, wherein generating, based on the first PET data and the second PET data, a target attenuation map of the subject includes:
generating, based on the first PET data and the second PET data, a preliminary attenuation map, the preliminary attenuation map being an attenuation map of the subject with respect to gamma rays with a first energy level; and
obtaining the target attenuation map based on the preliminary attenuation map and a conversion coefficient, the target attenuation map being an attenuation map of the subject with respect to gamma rays with a second energy level.

3. The method of claim 2, wherein generating, based on the first PET data and the second PET data, a preliminary attenuation map includes:
generating a first sinogram based on the first PET data;
generating a second sinogram based on the second PET data;
generating a difference sinogram between the first sinogram and the second sinogram; and
generating the preliminary attenuation map by performing an image reconstruction on the difference sinogram.

4. The method of claim 2, further including:
obtaining a first attenuation coefficient of a reference material with respect to gamma rays with the first energy level;
obtaining a second attenuation coefficient of the reference material with respect to gamma rays with the second energy level; and
determining, based on the first attenuation coefficient and the second attenuation coefficient, the conversion coefficient.

5. The method of claim 1, wherein generating, based on the first PET data and the second PET data, a target attenuation map of the subject includes:
generating first target PET data by performing a first noise reduction operation on the first PET data;
generating second target PET data by performing a second noise reduction operation on the second PET data; and
generating, based on the first target PET data and the second target PET data, the target attenuation map of the subject.

6. The method of claim 5, wherein at least one of the first noise reduction operation and the second noise reduction operation is performed based on a noise reduction model.

7. The method of claim 6, wherein the noise reduction model is generated using a plurality of training samples, and each of the plurality of training samples includes first sample PET data corresponding to a first acquisition duration and second sample PET data corresponding to a second acquisition duration, wherein the first acquisition duration and the second acquisition duration are different.

8. The method of claim 1, wherein the first particle includes a beta electron, the second particle includes a gamma photon.

9. The method of claim 1, wherein each coincident event of the first background events and the second background events is determined by:
obtaining the background coincident window, the background coincident window including a first energy window corresponding to the first particle, a second energy window corresponding to the second particle, and a time window;
determining whether the first particle and the second particle meet a predetermined condition based on a first energy of the first particle, a first detection time of the first particle, a second energy of the second particle, a second detection time of the second particle, and the background coincident window; and
in response to determining that the first particle and the second particle meet the predetermined condition, determining that the background event occurs.

10. The method of claim 1, further comprising:
obtaining third PET data relating to coincident events occur during a third PET scan, the third PET scan being performed by the PET scanner on the subject who has been injected with a radioactive tracer; and
generating, based on the target attenuation map and the third PET data, a PET image of the subject.

11. A system for attenuation map generation, comprising:
at least one storage device storing a set of instructions; and
at least one processor configured to communicate with the at least one storage device, wherein when executing the set of instructions, the at least one processor is directed to perform operations including:
obtaining first positron emission tomography (PET) data relating to first background events occur during a first PET scan, the first PET scan being performed by a PET scanner with no subject within a detection tunnel of the PET scanner;
obtaining second PET data relating to second background events occur during a second PET scan, the second PET scan being performed by the PET scanner with a subject within the detection tunnel; and
generating, based on the first PET data and the second PET data, a target attenuation map of the subject, wherein the first background events and the second background events occur when a first particle and a second particle are detected by crystal units of the PET scanner within a background coincident window, and the first particle and the second particle are different types of particles produced by a radioactive decay of a crystal material of the PET scanner.

12. The system of claim 11, wherein generating, based on the first PET data and the second PET data, a target attenuation map of the subject includes:
generating, based on the first PET data and the second PET data, a preliminary attenuation map, the preliminary attenuation map being an attenuation map of the subject with respect to gamma rays with a first energy level; and
obtaining the target attenuation map based on the preliminary attenuation map and a conversion coefficient, the target attenuation map being an attenuation map of the subject with respect to gamma rays with a second energy level.

13. The system of claim 12, wherein generating, based on the first PET data and the second PET data, a preliminary attenuation map includes:
generating a first sinogram based on the first PET data;
generating a second sinogram based on the second PET data;
generating a difference sinogram between the first sinogram and the second sinogram; and
generating the preliminary attenuation map by performing an image reconstruction on the difference sinogram.

14. The system of claim 12, the operations further including:
obtaining a first attenuation coefficient of a reference material with respect to gamma rays with the first energy level;
obtaining a second attenuation coefficient of the reference material with respect to gamma rays with the second energy level; and
determining, based on the first attenuation coefficient and the second attenuation coefficient, the conversion coefficient.

15. The system of claim 11, wherein generating, based on the first PET data and the second PET data, a target attenuation map of the subject includes:
generating first target PET data by performing a first noise reduction operation on the first PET data;
generating second target PET data by performing a second noise reduction operation on the second PET data; and
generating, based on the first target PET data and the second target PET data, the target attenuation map of the subject.

16. The system of claim 15, wherein at least one of the first noise reduction operation and the second noise reduction operation is performed based on a noise reduction model.

17. The system of claim 16, wherein the noise reduction model is generated using a plurality of training samples, and each of the plurality of training samples includes first sample PET data corresponding to a first acquisition duration and second sample PET data corresponding to a second acquisition duration, wherein the first acquisition duration and the second acquisition duration are different.

18. The system of claim 11, wherein each coincident event of the first background events and the second background events is determined by:
obtaining the background coincident window, the background coincident window including a first energy window corresponding to the first particle, a second energy window corresponding to the second particle, and a time window;
determining whether the first particle and the second particle meet a predetermined condition based on a first energy of the first particle, a first detection time of the first particle, a second energy of the second particle, a second detection time of the second particle, and the background coincident window; and
in response to determining that the first particle and the second particle meet the predetermined condition, determining that the background event occurs.

19. The system of claim 11, the operations further including:
obtaining third PET data relating to coincident events occur during a third PET scan, the third PET scan being performed by the PET scanner on the subject who has been injected with a radioactive tracer; and
generating, based on the target attenuation map and the third PET data, a PET image of the subject.

20. A non-transitory computer readable medium, comprising at least one set of instructions, wherein when executed by at least one processor of a computing device, the at least one set of instructions causes the computing device to perform a method, the method comprising:
obtaining first positron emission tomography (PET) data relating to first background events occur during a first PET scan, the first PET scan being performed by a PET scanner with no subject within a detection tunnel of the PET scanner;
obtaining second PET data relating to second background events occur during a second PET scan, the second PET scan being performed by the PET scanner with a subject within the detection tunnel; and
generating, based on the first PET data and the second PET data, a target attenuation map of the subject, wherein the first background events and the second background events occur when a first particle and a second particle are detected by crystal units of the PET scanner within a background coincident window, and the first particle and the second particle are different types of particles produced by a radioactive decay of a crystal material of the PET scanner.
